(19) United States
(12) Patent Application Publication
(10) Pub. No.: US 2017/0281953 A1
(43) Pub. Date: Oct. 5, 2017

(54) LEADLESS SPINAL CORD STIMULATION SYSTEM AND METHOD INCLUDING SAME

(71) Applicant: PACESETTER, INC., Sylmar, CA (US)

(72) Inventors: Xiaoyi Min, Camarillo, CA (US); John W. Poore, South Pasadena, CA (US)

(21) Appl. No.: 15/628,531

(22) Filed: Jun. 20, 2017

Related U.S. Application Data
(63) Continuation of application No. 14/221,151, filed on Mar. 20, 2014, now abandoned.

(51) Int. Cl. A61N 1/375

(52) U.S. Cl. CPC A61N 1/3756; A61N 1/37288

(57) ABSTRACT

A leadless neurostimulation (NS) device and method to manufacture the device is described. The leadless NS device has a first sub-unit (FU) and a second sub-unit (SU) separately and individually hermetically sealed. The FU and SU also include a flexible inter-connect that physically interconnects the FU and SU to one another. The leadless NS device also includes electrodes provided along the exterior surface of at least one of the first and second sub-units. The electrodes are configured to interface with nervous tissue in an epidural space of a patient and deliver stimulation pulses along the nervous tissue. At least partially housed within the FU includes a first subset of a power source, an energy management components, an electronics sub-system and telemetry component. Further, a second subset of the power source, energy management components, electronics sub-system and telemetry component are at least partially housed within the SU.

### CROSS REFERENCE TO RELATED APPLICATIONS

This application is a continuation of U.S. patent application Ser. No. 14/221,151, filed Mar. 20, 2014.

### BACKGROUND OF THE INVENTION

Embodiments of the present disclosure generally relate to neurostimulation (NS) systems generating electric pulses proximate to nervous tissue, and more particularly to spinal cord stimulation (SCS) systems.

NS systems are configured to generate electrical pulses and deliver the pulses to nervous tissue to treat a variety of disorders. SCS is a common type of neurostimulation. In SCS, electrical pulses are delivered to nervous tissue in the spine to generate electric pulses that can treat a neurologic condition. For example, the application of an electric pulse to spinal nervous tissue can effectively mask or alleviate certain types of pain transmitted from regions of the body associated with the stimulated nervous tissue.

Conventional NS systems may include a pulse generator and one or more elongated leads that are electrically coupled to the pulse generator. Each elongated lead includes a stimulating end, a trailing end, and an intermediate portion that couples the stimulating and trailing ends. The elongated lead may be cable-like and extend, for example, up to sixty centimeters or more between the stimulating and trailing ends. The stimulating end may have a body with multiple electrodes that are configured to interface with nervous tissue, such as within an epidural space of a spinal cord. The trailing end includes multiple terminal contacts that engage corresponding contacts of the pulse generator. The terminal contacts of the trailing end and the electrodes of the stimulating end are coupled by wire conductors that extend through the intermediate portion. In use, the pulse generator controls current through the wire conductors to generate the electric pukes along the nervous tissue. The puke generator is typically implanted within the patient in a subcutaneous pocket formed near the surface of the skin. The puke generator may be programmed (and re-programmed) to provide the electrical pukes in accordance with a designated sequence.

Typically, one of two types of leads is used. The first type is a percutaneous lead, which has a rod-like shape and includes electrodes spaced apart from each other along a single axis. The second type of lead k a laminectomy or laminotomy lead (hereinafter referred to as a paddle lead). A paddle lead may have an elongated and generally planar body with a substantially rectangular shape (i.e., paddle-like shape). Paddle leads typically include an array of electrodes that are spaced apart from each other. The number of electrodes may be, for example, four, eight, sixteen, or more.

Although such NS systems can be effective for treating one or more neurologic conditions, some drawbacks or challenges may exist. For example, NS systems may be prone to heating and induced currents when placed within strong gradient and/or radiofrequency (RF) magnetic fields of a magnetic resonance imaging (MRI) system. The heat and induced currents result from the metal components of the leads functioning as antennas in the magnetic fields. Components of the system may also move due to the force/torque generated in the static magnetic field of an MRI system.

in addition to the above, the number of components and overall shape and size of a conventional NS system may increase the likelihood of infection or require a follow-up surgery for the patient. For instance, in order to implant the entire NS system, the elongated lead k tunneled from the epidural space through the body and into the subcutaneous pocket where the pulse generator is located. NS systems that do not require tunneling and a subcutaneous pocket may reduce the likelihood of infection and/or a follow-up procedure being necessary.

### SUMMARY

In accordance with one embodiment, a leadless neurostimulation (NS) device is described with first and second sub-units separately and individually hermetically sealed relative to one another. Each of the first and second sub-units has an exterior surface configured to be implantable proximate to a spinal column of a patient. The leadless NS device further has electrodes along the exterior surface of at least one of the first and second sub-units. The electrodes are configured to interface with nervous tissue in an epidural space of a patient and deliver stimulation pulses along the nervous tissue. Further, the leadless NS device includes a power source and an energy management components electrically coupled to the power source and a telemetry component configured to communicate with a device external to the patient.

Additionally, the leadless NS device includes an electronics sub-system with a controller and a switching circuitry. The controller and switching circuitry are configured to control delivery of the stimulation pulses through the electrodes. At least partially housed within the first sub-unit is a first subset of the power source, energy management components, electronic sub-system and telemetry component. Also, a second subset of the power source, energy management components, electronic subsystem and telemetry component are at least partially housed within the second sub-unit. Further, a flexible inter-connect physically interconnects the first and second sub-units to one another and electrically interconnects the power source, energy management components, electronics sub-system and telemetry component.

In an embodiment, a method of manufacturing a leadless neurostimulation (NS) device to be implantable proximate to a spinal column of a patient is provided. The method includes providing hermetically sealed first and second sub-units. The first and second subunits include a power source and an energy management components coupled to the power source. The method further includes positioning at least one electrode along the exterior surface of at least one of the first and second sub-units. The electrode is configured to generate an electric pulse in an outward radial direction proximate to nervous tissue. The method also includes coupling the electrode to switching circuitry. The switching circuitry is configured to electrically set a state of the electrode. The method further includes providing a control unit in at least one of the first and second sub-units. The control unit is configured to execute a protocol determining the state of the electrode. And the method includes interconnecting the first and second sub-units with a flexible inter-connect. The flexible inter-connect provides a single conductive path feed-through located at a first end of the first sub-unit and a first end of the second sub-unit. The single conductive path feed-through is configured to carry at least two of a device power, communication data, and/or stimulation pulses between the first and second sub-units.

### BRIEF DESCRIPTION OF THE DRAWINGS

FIG. 1 illustrates a side view of a neurostimulation (NS) system for applying electric pulses to nervous tissue of a patient in accordance with one embodiment.

FIG. 2 is a schematic diagram of an NS device in accordance with one embodiment, which may be used with the NS system of FIG. 1.

FIG. 3a is a graphical representation of a signal carried along the flexible inter-connect.

FIG. 3b is a graphical representation of a signal partitioned from the signal in FIG. 3a.

FIG. 3c is a graphical representation of a supply voltage partitioned from the signal in FIG. 3a.

FIG. 4 is an alternative schematic diagram of the NS device in FIG. 1.

FIG. 5 is a schematic diagram of an NS device in accordance with one embodiment, which may be used with a NS system.

FIG. 6 is an electrical diagram of a cell used for controlling the output to an electrode.

FIG. 7 illustrates an electrical diagram for generating electric pulses proximate to nervous tissue in accordance with an embodiment.

FIG. 8 illustrates another electrical diagram for generating electric pulses proximate to nervous tissue in accordance with an embodiment.

FIG. 9 illustrates a schematic diagram of a flexible interconnect of a sub-unit of a leadless NS device.

FIG. 10 is a flowchart illustrating a method of manufacturing a NS device.

### DETAILED DESCRIPTION

Embodiments described herein include neurostimulation (NS) leads, NS systems, and methods of manufacturing or using the same. The NS device may he configured to be inserted into a space or cavity of a patient and positioned adjacent to nervous tissue. In certain embodiments, the NS device includes wireless leads that are positioned entirely within an epidural space of a spinal column. The NS devices may include sub-units having a length within the size of vertebral bone separately and individually hermetically sealed relative to one another. The implantable sub-units may include an electronic sub-system (or pulse, generator) and an array of electrodes operably coupled to the electronic sub-system. The implantable device sub-units are physically interconnected with a flexible inter-connect which electrically interconnects the electronic sub-systems of each sub-unit. The electronic sub-system may include, for example, a controller and switching circuitry. The electronic sub-system is configured to generate electric pulses with the electrodes for providing a therapeutic stimulation. In particular embodiments, the electronic sub-system interacts with a telemetry component that uses inductive coupling to communicate with external devices to the patient. For instance, embodiments may interact through inductive coils, which may also be referred to as primary or secondary cons depending upon the function of the coil. During operation, the primary and secondary coils may at least one of communicate data (e.g., pulse data) or transmit/receive electrical power.

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

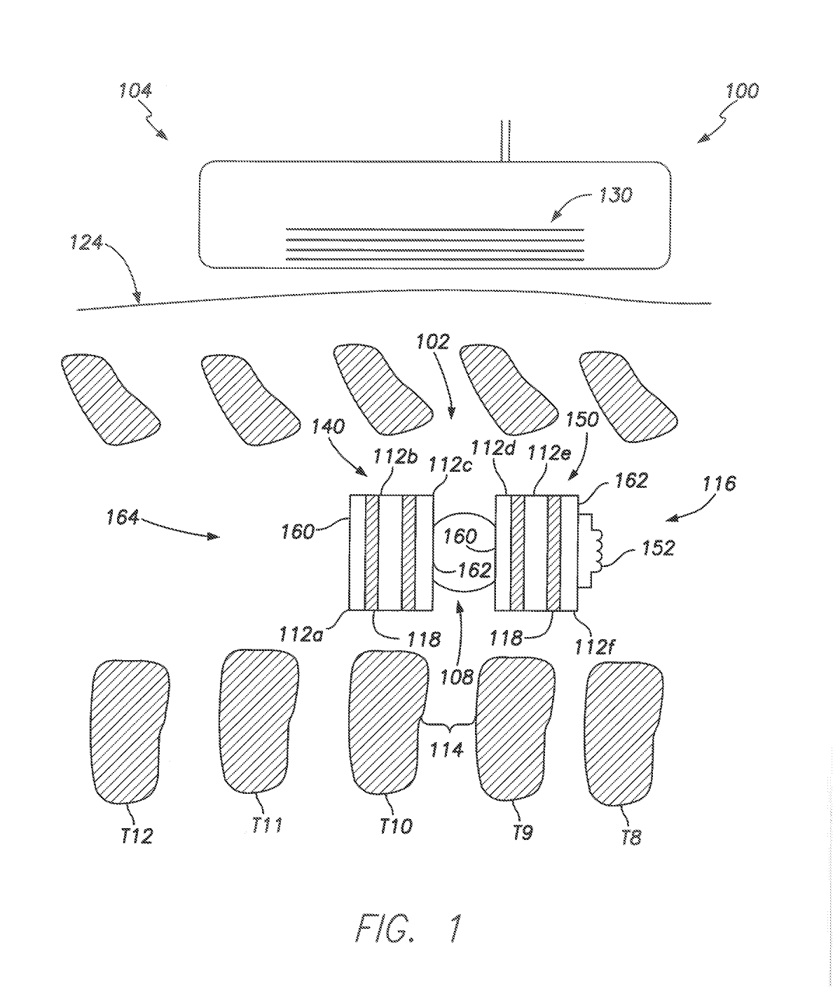

FIG. 1 depicts a NS system 100 that includes an implantable NS device 102 and an external monitoring system 104, near a skin surface 124 of the patient, configured to communicate with and power (e.g., charge) the NS device 102. The NS device 102 may have first and second sub-units 140 and 150 that are implantable and proximate to the spinal column of a patient. The sub-units 140 and 150 extend along a longitudinal axis 164 parallel to the spinal column. In the illustrated embodiment, the first and second sub-units 140 and 150 have a length that is sized to fit within vertebral bone T10 and T9, respectively. Optionally, in other embodiments the sub-unit length of the first sub-unit 140 and/or the second sub-unit 150 may be greater or smaller than a vertical height of a vertebral bone. For example only, the first and second sub-units 140 and 150 may have a sub-unit length from opposite first and second ends 160 and 162 of approximately 24 millimeters (mm) extending from end 160, proximate to vertebra T11, to the opposite end 162, proximate to vertebra T8.

The first and second sub-units 140 and 150 interface with nervous tissue (e.g., dorsal column (DC) fibers and/or dorsal root (DR) fibers) of a patient within an epidural space 116 proximate to a spinal column. The first and second sub-units 140 and 150 are each separately and individually hermetically sealed. The nervous tissue engaged by the first and second sub-units 140 and 150 include the spinal cord along the thoracic vertebra T10 and T9, respectively. The first and second sub-units 140 and 150 interface with the dura mater of the spinal column. The cerebrospinal fluid and nerve fibers, which are surrounded by the dura mater, are not shown in FIG. 1. However, it is noted that FIG. 1 shows only one application of the NS device 102. It is understood that embodiments may be used in other NS applications.

The sub-units 140 and 150 may have a cylindrical shape, an oval shape, a disk shape, a paddle shape, or the like. It should be noted that the NS device 102 may have more (as described below) or less sub-units than illustrated in FIG. 1. The first and second sub-units 140 and 150 include a plurality of electrodes 112. The electrodes 112a-f may be in the shape of a ring such that each electrode 112a-f continuously covers the circumference of the exterior surface of the sub-unit 140 and 150. The electrodes 112a-f are separated by non-conducting rings 118, which electrically isolate each electrode 112a-f from an adjacent electrode 112. The non-conducting rings 118 may include one or more insulative materials and/or biocompatible materials to allow the NS device 102 to be implantable proximate to the spinal column of the patient. Non-limiting examples of such materials include polyimide, polyetheretherketone (PEEK), polyethylene terephthalate (PET) film (also known as polyester or Myler), polytetrafluoroethylene (PTFE) (e.g., Teflon), or parylene coating, polyether bloc amides, polyurethane. The electrodes 112a-f are configured to emit current or generate electric pulses in an outward radial direction proximate to the nervous tissue. Optionally, one or more of the electrodes 112a-f may be segmented circumferentially in two or more arc segments. Each of the arc segments may be electrically insulated from each other, allowing directional electrical stimulation towards tissue proximate to the surface area of the arc segment. Further, segmenting the electrodes 112a-f may increase the efficiency of the nervous tissue stimulation of the NS device by reducing the proportion of electrical pulses from the electrodes 112a-f delivered to non-nervous tissue. It should be noted that each of the first and second sub-units 140 and 150 may have more or less (e.g., zero) electrodes 112a-f than the number of electrodes 112a-f (e.g., three) illustrated in FIG. 1.

A flexible inter-connect 108 made of, for example, silicone, polyurethane, or the like physically interconnects the first and second sub-units 140 and 150 at the distal end of the first sub-unit 140, proximate to T9, and the proximal end of the second sub-unit 150, proximate to T10. Optionally, the flexible inter-connect 108 may be aligned with the sub-units 140 and 150 along a common axis such as the longitudinal axis 164. The flexible inter-connect 108 is shown being slightly larger than a gap 114 between the vertebral bone allowing the first and second sub-units 140 and 150 to be positioned within the respective vertebral bones T9 and T10. It should be noted that the flexible inter-connect 108 may be larger or smaller than illustrated in FIG. 1. For example only, the vertebral bones T9 and T10 have a width or size of 25 mm each separated by the gap 114 of 1.5 mm. The first and second sub-units 140 and 150 have a sub-unit length of 22 mm and 24 mm, respectively, and the flexible inter-connect 108, physically interconnecting the sub-units 140 and 150 has a length of at least 5 mm. The length of the flexible inter-connect 108, being greater than the gap 114, creates a buffer or allowance of movement of the sub-units 140 and 150. The buffer allows the NS device to flex with the vertebral bones T10 and T9, and for the NS device to remain within the vertebral bones T10 and T9, respectively, while the patient, spinal cord, or sub-units 140 and 150 shift.

Additionally, the flexible inter-connect 108 may be compressed, reducing a distance between the first and second sub-units 140 and 150, during implantation of the NS device 102 in the delivery tool (e.g., a catheter) and expanded by the delivery tool when in a selected position inside epidural space 116.

The NS device 102 may interact with the monitoring system 104. For example, the monitoring system 104 and the NS device 102 may communicate, with each other, one or more times after the NS device 102 has been implanted. At later intervals (e.g., once a week, twice a month, once every two months, and the like), the monitoring system 104 and the NS device 102 may interact with each other to (i) communicate data between the NS device 102 and the monitoring system 104 and/or (b) charge the power source 206.

To this end, the monitoring system 104 and the NS device 102 may include telemetry components 130 and 152, respectively (e.g., an inductive coil, Bluetooth transmitter/receiver, Zigbee transmitter/receiver, or the like). The telemetry component 130 may be referred to as a primary telemetry, and the telemetry component 152 shown coupled to the second sub-unit 150 may be referred to as a secondary telemetry. The telemetry component 130 may be sized and shaped to be larger than the telemetry component 152. In some embodiments, the telemetry components 152, 130 may (a) communicate data for operating and monitoring conditions of the NS device 102 in the patient and (b) electrically power or charge the NS device 102. In other embodiments, however, at least one of the monitoring system 104 or the NS device 102 may include more than one inductive coil (e.g., an inductive coil on both the first and second sub-units 140 and 150) in which each inductive coil has separate functions. For example, one telemetry component may be used to communicate data and another telemetry component may be used to transmit/receive electrical power.

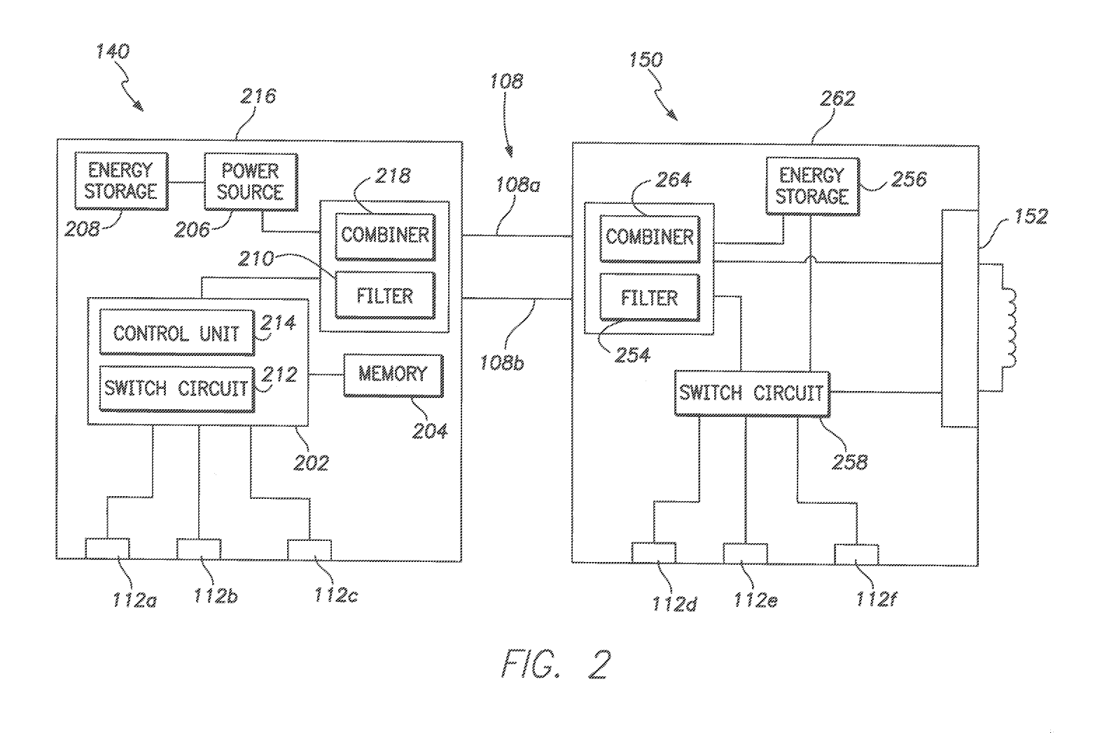

FIG. 2 is a schematic diagram illustrating components of an embodiment of the first and second sub-units 140 and 150 of the NS device 102 (FIG. 1) each within individual cans (or housings) 216 and 262. The sub-units 140 and 150 include electronic sub-systems 202, which may include one or more neurostimulation (NS) controller unit 214 and one or more switching circuits 212 and 258, respectively. The switching circuits 212 and 258 may also be characterized as switch arrays, switch matrixes, or multiplexers/de-multiplexers that are coupled to the plurality of electrodes 112, such that the switching circuit 212 may activate or control the electrodes 112a-f separately or independently for the respective sub-units 140 and 150. The electronic sub-system 202 is configured to control operation of the sub-units 140 and 150, and interact with the alternative sub-units 150 and 140 through the flexible inter-connect 108.

The control units 214 may control both switching circuitries 212 and 258 and other electronics to generate electric pulses at a select current in accordance with parameters specified by one or more neurostimulation parameter sequences (or protocols) stored within the memory 204. Exemplary parameters for the electrical pulses may include a puke amplitude, pulse width, and pulse rate for a stimulation waveform. Additionally, the control unit 214 may control the switching circuitry 212 and 258, to select different electrode configurations or states for generating the designated electric pukes. For example, the control unit 214 may instruct the switching circuitries 212 and 258 to set one or more of the electrodes 112a-f, respectively, to an anode state (e,g., couple the selected electrodes 112a-f to the voltage supply, the power source, or the energy management components 208 and 256), a cathode state (e.g., a sink), and/or an inoperative electrode state (in which case the electrode is not used for transmitting energy, i.e., is inactive or open).

The electric sub-system 202 is coupled to one or more current/voltage sources. The control unit 214 may control the current/voltage sources to deliver a single stimulation puke or multiple stimulation pukes. In some embodiments, the current/voltage source and the switching circuitry 212 and 258 may be configured to deliver stimulation pukes to multiple channels on a time-interleaved bask, in which case the switching circuitry 212 and 258 may time division multiplex the output of current/voltage source across different combinations of electrodes 112a-f at different times to deliver multiple pukes or therapies to the patient.

In some embodiments, the implementation of the components within the sub-units 140 and 150 of the NS device 102 set forth herein, such as the control unit 214, current/voltage sources, memory 204, and switching circuitry 212 and 258 may be similar to or function in a similar manner as the components described in U.S. Patent Application Publication No. 2006/0259098, entitled “SYSTEMS AND METHODS FOR USE IN PULSE GENERATION,” which is incorporated herein by reference in its entirety. One or more of the sub-units 140 and 150 may have an exterior surface with electrodes similar to paddle leads described in U.S. Patent Application Publication No. US 2013/0006341, which is incorporated herein by reference in its entirety.

Control circuitry (e.g., electric sub-system 202, snitching circuitry 212 and 258, control unit 214) may be constructed as described in the U.S. Patent Application Publication No. 2006/0170486 entitled “PULSE GENERATOR HAVING AN EFFICIENT FRACTIONAL VOLTAGE CONVERTER AND METHOD OF USE,” which is incorporated herein by reference in its entirety. One or multiple sets of the control circuitry may be provided within the sub-units 140 and 150 of the NS device 102. Different pulses on different electrodes 112a-f may be generated using a single set of pulse generating circuitry using consecutively generated pulses according to a “multi-stimset program.” Complex pulse parameters may be employed such as those described in U.S. Pat. No. 7,228,179, entitled “METHOD AND APPARATUS FOR PROVIDING COMPLEX TISSUE STIMULATION PATTERNS ” and International Patent Publication No. WO 2001/093953 A1 entitled “NEUROMODULATION THERAPY SYSTEM” each of which is incorporated herein by reference in its entirety. Although constant current pulse generating circuitry is contemplated for some embodiments, any other suitable type of pulse generating circuitry may be employed such as constant voltage pulse generating circuitry.

The control unit 214 may control operation of the sub-units 140 and 150, pursuant to designated stimulation protocols. A single control unit 214 controls the stimulation protocols of multiple sub-units (e.g., sub-units 140 and 150) through the flexible inter-connect 108, discussed further below. Each stimulation protocol may include one or more sets of stimulation parameters including puke amplitude, pulse width, puke frequency or inter-pulse period, pulse repetition parameter (e.g., number of times for a given pulse to be repeated for respective stimset during execution of program), etc. NS systems, stir sets, and multi-stimset programs are discussed in PCT Publication No. WO 01/193953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

The sub-units 140 and 150 also include at least one power source 206 used to provide or distribute power to the internal circuitry of the respective sub-units 140 and 150 such as the electrical sub-system 202 and supplying power for transmitting electrical pulses during neurostimulation. The power from the power source 206 may be used to charge energy management components 208 and 256 coupled to the power sources 206. In some embodiments, the power source 206 may be a rechargeable power source, such as a lithium ion rechargeable (LIR) battery. By way of example only, the capacity of the power source 206 may be from about 20 mAh to about 180 mAh with a nominal voltage of about 3.60V. Examples of suitable LIR batteries includes Eagle Richer LIR 2025, 2430, 2450 and the like or Quallion QL0003I. In some embodiments, the power source 206 may be capable of operating between one week to one month (or more) between charges with about a 100-150 μA stimulation current drain,

The energy management components 208 and 256 may be one or more capacitors, inductors, FETs, and/or the like. Additionally or alternatively, the energy management components 208 and 256, may be configured to increase or decrease available voltage or current capabilities of the battery, for example, using a single capacitor or having an inductor, capacitor, and FET in a boost configuration. The charge or current stored within the energy management components 208 and 256 may be dissipated by the electrodes 112a-f via the switching circuitry 212 and 258. For example, the energy management components 208 may include a capacitor coupled to the power source 206 (e.g., LIR battery). The power source 206 applies power to the energy management components 208 (e.g., capacitor) to charge the energy management components 208.

The control unit 214 controls the switching circuitries 212 and 258 to connect a select combination of the electrodes to the energy management components 208 and 256. For example, the control unit 214 instructs the switching circuitry 212 to electrically couple the energy management components 208 to the electrodes 112a-c (e.g., an anode state). The control unit 214 may further instruct the switching circuitry 258 to set electrode 112d to a cathode state (e.g., a sink), and electrodes 112e-f to an inoperative electrode state (in which case the electrode is not used for transmitting energy, i.e., is inactive or open). The charge or voltage stored on the energy management components 208 is dissipated from the electrodes 112a-c as current traveling to the electrode 112d.

At least one of the sub-units 140 and 150 may include an inductive coil 150. The telemetry component 152 (e.g., inductive coil) is operatively coupled (via the flexible inter-connect 108) to the control unit 214. The telemetry component 152 may transmit data and other information from the sub-units 140, 150 to the monitoring system 104. Additionally or alternatively, the telemetry component 152 may be operatively coupled to a power source. In some embodiments, the telemetry component 152 is configured to receive signals (e.g., instructions or other data) from the monitoring system 104 (FIG. 1) and communicate the signals to the control unit 214. The signals may include, for example, software updates, updated stimulation sequences, data regarding conditions of the NS device 102 or the patient, and the like. The telemetry component 152 may also be configured to receive charging electrical power from the monitoring system 104 and transfer the electrical power to the power source 206 through the flexible inter-connect 108. Circuitry for recharging the power source of the NS device using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference in its entirety.

The flexible inter-connect 108 electrically interconnects the internal components of the first and second sub-units 140 and 150. For example, the inter-connect 108 connects one or more the power source 206, electronic sub-systems 202, energy management components 208 and 256, and telemetry components 152. The inter-connect 108 is joined to each sub-unit 140 and 150 at a corresponding feedthrough. Each conductive path feed-through 108a defines a conductive path into and out of the corresponding sub-unit 140 and 150, carrying electric charges or signals (e.g., communication, device power, stimulation pulses) that enter or leave the hermetically sealed interior of the corresponding sub-unit 140 and 150. For example, the energy management components 208 may be a capacitor. The conductive path feed-through 108a carries or distributes a supply voltage from the power source 206 (LIR battery) in one sub-unit 140 to the capacitor in another sub-unit 150.

Additionally or alternatively, the flexible inter-connect 108 may include a common ground 108b which may be used as a reference for the electric charge (e.g., voltage) carried with the conductive path feed-through 108a that enters or leaves the hermetically sealed interior of the corresponding sub-unit 140 and 150. It should be noted that the flexible inter-connect 108 may be a single wire or cable (e.g., coaxial cable). For example, the flexible inter-connect 108 may be a single cable with the conductive path feed-through 108a as an inner wire surrounded by the common ground 108b separated by an insulator (e.g., dielectric insulator), which isolates an electric field or signal of the conductive path feed-through from the common ground 108b.

At each end of the flexible inter-connect 108, within the sub-units 140 and 150, the flexible inter-connect 108 may be electrically coupled to power/data filters 210 and 254 and power/data combiners 218 and 264. The power/data filters 210 and 254 separate or partition a signal (e.g., communication data, inter-module control signals, etc.) embedded or atop of the electrical signal propagated by the flexible inter-connect 108. The power/data filters 210 and 254 may include comparators, a feedthrough capacitor, decoder, endec, or the like.

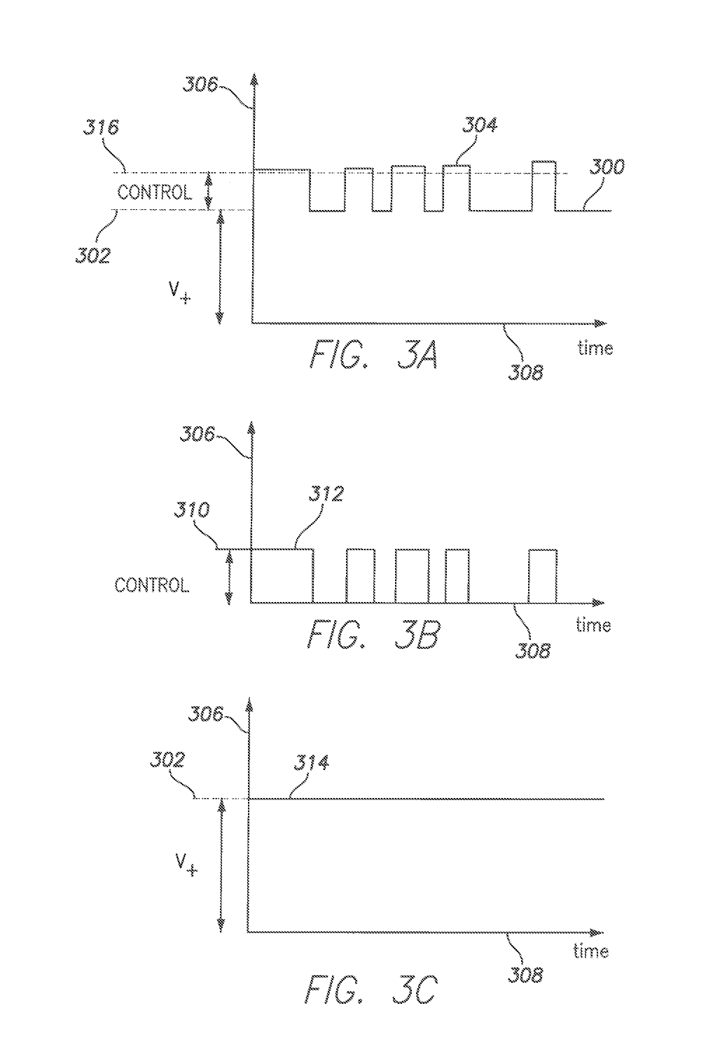

For example, FIG. 3a illustrates a graphical representation of a signal 300 carried along the flexible inter-connect 108, and FIGS. 3b and 3c are graphical representations of two signals 312 and 314 partitioned from the signal 300. Horizontal axes 308 represent time and vertical axes 306 represent a voltage. Additionally or alternatively, the vertical axes 306 represent a difference in electrical potential (e.g., voltage.) of the conductive path feed-through 108a and the common ground 108b. The voltage, V+ 302, represents the supply voltage used by the internal components of the sub-units 140 and 150. An embedded signal 312 may be added to V+ 302 illustrated as bits or rectangular pulses with a peak voltage 304 in FIG. 3a. It should be noted that the embedded signal 312 may be in a form or wave other than illustrated in FIG. 3a such as an analog or sinusoidal wave. A predetermined voltage threshold 31$ may be used by the power/data filter 210 and 254 to distinguish the embedded signal 312 from the supply voltage, V+ 302. The embedded signal 312 may represent communications data received from the external monitoring system 104 via the telemetry component 152 or one of the sub-units (e.g., 140, 150).

FIG. 3b illustrates the embedded signal 312 partitioned from the signal 300 of FIG. 3a. The embedded signal 312 has an amplitude 310, which may be the approximate difference between the predetermined voltage threshold 316 and the supply voltage, V+ 302. Once partitioned by the power/data filter 210 and 254, the embedded signal 312 may be received by the electric subsystem 202 and/or the switching circuitry 258.

For example, the control unit 214 may compare the embedded signal 312 with a predetermined protocol stored on the memory 204 based on the content (e.g., sequence of bytes) of the embedded signal 312. The protocol may represent possible instruction states (e.g., charge the power source 206) received by the monitoring system 104 through the telemetry component 152. Optionally, the control unit 214 may compare the embedded signal 312 with a predetermined address sequence matching the NS device 102 sub-unit. The control unit 214 may determine from the predetermined protocol whether the embedded signal 312 includes communication data from another sub-unit such as from switching circuitry 258 of the sub-unit 150, the telemetry component 152 (e.g., begin communication with the monitoring system 104, or the energy management components 256). Additionally or alternatively, the communication data may include NS device 102 or patient status information from another sub-unit for transmission to an external device (e,g., the monitoring system 104). In an embodiment, the communication data may include external telemetry equipment data (e.g., stimulation parameters, protocol update) from the telemetry component 152 transmitted from an external device (e.g., the monitoring system 104) to the control unit 214.

Additionally, the embedded signal 312 may represent inter-module control signals (e.g., sub-unit electrode state instructions, energy management components states, telemetry component states, or the like) or switch states from the control unit 214 destined for the switching circuitry 258 or other components of the external sub-unit 150. For example, the embedded signal 312 may represent an instruction or switch state by the control unit 214 of the sub-unit 140 for the energy management components 256 of the sub-unit 150 to enter a charge state (electrically coupling the energy component 256 to the supply voltage, V+ 302). The instruction may be received by the switching circuitry 258, which electrically couples the energy management components 256 to the power/data filter 210 to receive the supply voltage. Additionally or alternatively, the switching circuitry 258 may supply the supply voltage received from the power/data filter 210 to the energy management components 256.

In an embodiment, the embedded signal 312 may represent an instruction to the switching circuitry 258 by the control unit 214 to enter a status or monitor state. The status or monitor state may be a measurement request or notification request by the control unit 214 on the current voltage or charge of the energy management components 256 or whether the energy management components 256 has a predetermined charge level for discharge (e.g., through the electrodes 112d-f). Additionally or alternative, the status state may represent a confirmation request by the control unit 214 on the current state of the electrodes 112d-f Optionally, the status or monitor state may instruct the switching circuitry 258 to couple the telemetry component to the power/data combiner 264 to broadcast or transmit a message to an external device (e.g., monitoring system 104).

Optionally, the inter-module control signal may be based on a frequency-division multiplexing scheme, such that, the embedded signal 312 may include multiple control signals each within a subset frequency of the total bandwidth of the embedded signal 312. The switching circuitry 258 may include multiple band pass filters corresponding to different switch states or components within the sub-unit 150. For example, the inter-module control signal for the electrode states may be within 100-125 Hertz (Hz), the energy management components states may be within 75-100 Hz, the telemetry component states may be within 50-75 Hz.

FIG. 3c illustrates the device power signal 314 partitioned from the signal 300 of FIG. 3a. The device power signal 314 is supplied by the power source 206. The device power signal 314 may have a voltage of V+ 302. Once partitioned by the power/data filter 254, the device power signal 314 may be received by the switching circuitry 258, which may distribute the device power signal 314 to different components of the sub-unit 150 as instructed by the control unit 214 (via the embedded signal 312).

Additionally or alternatively, the device power signal 314 may include telemetry power or battery charging energy from the telemetry component 152 to charge the power source 206. For example, the monitoring system 104 may transmit battery charging energy to the NS device 102. The battery charging energy is received by the telemetry component 152, which is detected by the switch circuitry 258. Optionally, the switch circuitry 258 may monitor the output of the telemetry component 152 for incoming transmissions using an edge-trigger to detect a rising edge of the transmission or a relay. The switch circuitry 258 may couple the output of the telemetry component 152 to the power/data combiner 264 to be received by the power source 206 to recharge.

The power/data combiner 218 and 264 combines or embeds the device power signal 314 with the embedded signal 312 (e.g., communications data signal, inter-module control signals) forming the signal 300, which is carded by the flexible inter-connect to other sub-units 140 and 150. The power/data combiner 218 and 264 may include an encoder, multiplier, adder or the like. Optionally, the power/data combiner 218 and 264 may include a modulation component. The modulation component modulates the current and/or voltage of the device power signal 314 by superimposing the communications data and/or inter-module control signals onto the device power signal 314 as illustrated in FIG. 3a.

As shown, the control unit 214, the power source 206, the memory 204, the switching circuitry 212 and 258, the power/data combiner 218 and 264, and the power/data filters 210 and 254 are illustrated as separate blocks. It is understood, however, that such distinctions are not necessarily indicative of the division between hardware circuitry. Thus, for example, one or more of the functional blocks (e.g., control unit 214, switching circuitry 212, memory 204, the power/data combiner 218, the power/data filer 210) may be implemented in a single piece of hardware or through multiple pieces of hardware. The electronic sub-system 202 and its components may control the various modes for providing stimulation therapy and, optionally, monitoring such stimulation therapy. More specifically, it is to be understood that the different functions or operations described herein that are performed by the electronic sub-system 202 and its components (e.g., the control unit 214 and the switching circuitry 212) may be implemented using hardware with associated instructions (e.g., software stored on a tangible and non-transitory computer readable storage medium, such as a computer hard drive, ROM, RAM, or the like) that perform the functions/operations described herein. The hardware may include state machine circuitry hard wired to perform the functions/operations described herein. Optionally, the hardware may include electronic circuits that include and/or are connected to one or more logic-based devices, such as microprocessors, processors, controllers, or the like. Optionally, the components may include processing circuitry such as one or more field programmable gate array (FPGA), application specific integrated circuit (ASIC), system on chip (SoC), or microprocessor. The components in various embodiments may be configured to execute one or more algorithms to perform functions described herein. The one or more algorithms may include aspects of embodiments disclosed herein, whether or not expressly identified in a flowchart or a method.

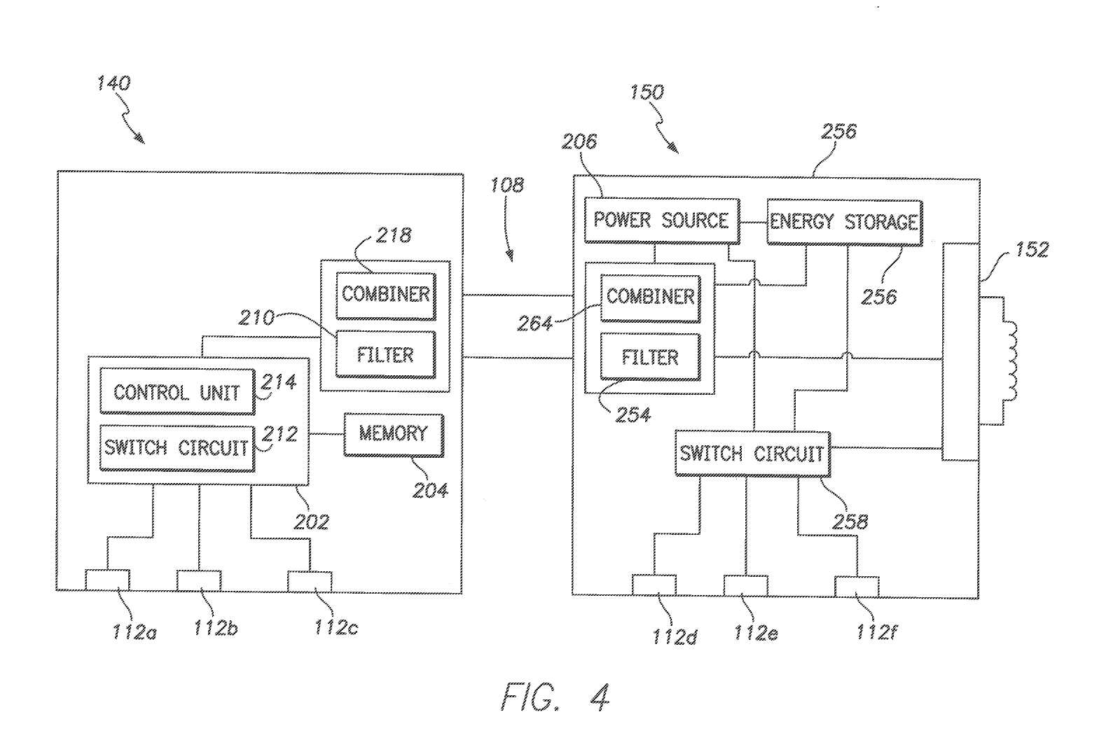

It should be noted that there are other possible configurations of the sub-units 140 and 150 and the respective internal components (the control unit 214, the switching circuitry 212 and 258, the power source 206, the electrodes 112a-f, the telemetry component 152, the energy management components 208) than illustrated in FIG. 2. For example, FIG. 4 is a schematic diagram of an alternative exemplary embodiment 102a of the NS device 102 illustrated in FIG. 2. The sub-unit 150 is in a slave or dependent configuration with the sub-unit 140 due to the location of the control unit 214 within the sub-unit 140. The operation of the sub-unit 150 (e.g., the switching circuitry 258, the telemetry component 152, the electrodes 112d-f). The sub-unit 150 receives configuration instructions or commands from the control unit 214 through the flexible inter-connect 108. The sub-unit 140 does not include a telemetry component (e.g., inductive coil, wireless transmitter). The control unit 214 transmits/receives communication data from an external device (e.g., the monitoring device 104) using the telemetry component 152 of the sub-unit 150 via the flexible inter-connect 108. The sub-unit 140 is remote power dependent on the sub-unit 150 due to the location of the power source 206 within the sub-unit 150. The sub-unit 140 receives supply voltage or power remotely from the power source 206 of the sub-unit 150 through the flexible inter-connect 108. Further, the energy management components 208 is not within the sub-unit 140, and may be shared among the two sub-units 140 and 150 through the flexible inter-connect 108.

For example, the control unit 214 outputs an instruction or switch state that instructs the switch circuitry 258 to discharge the energy management components 256 through the flexible inter-connect 108. The control unit 214 outputs the instruction to the power/data combiner 218 as an embedded signal 312. The power/data combiner 218 combines the embedded signal 312 with the device power signal 314 supplied by the power source 206. The flexible inter-connect 108 carries the signal 300 to the sub-unit 150. The power/data filter 254 receives the signal 300 and partitions the embedded signal 312, which is received by the switch circuitry 258. The switching circuitry 258 electrically couples the energy management components 256 to the device power signal 314 via the power/data filter 210 and decouples the power source 206. The control unit 214 also instructs the switching circuitry 212 to set the electrodes 112a-c to an anode state. The switching circuitry 212 couples the electrodes 112a-c to the power/data filter 210, which discharges the energy management component 256. Once discharged, the control unit 214 instructs the switching circuitry 212 to decouple the electrodes 112a-c from the power/data filter 210, and instruct switching circuitry 258 to electrically couple the power source 206 to the power/data combiner 264.

The NS device 102 sub-units 140 and 150 each comprise different functional combinations or “subsets” of the basic functional sub-systems (e.g., the power source 206 (FIG. 2), components, subsystems and other circuitry and structure) utilized to form a complete and functional NS device. By way of example, a complete and functional NS device 102 may include functional sub-systems for at least the power source 206 (fixed or rechargeable), energy management components 208 and 264, telemetry components 152, and electronics. The electronics include various structures such as, but not limited to, memory 204, switches, amplifiers, and the NS control unit 214. The control unit 214 may comprise circuitry or logic that is hard-wired to operate as a state machine and/or a microprocessor to operate based on software and/or firmware, switching components, amplifiers, filters, etc. The electronics may perform various sensing functions (e.g., to monitor physiologic states or behavior of interest, to monitor status of electrodes), stimulation functions (e.g., to delivery one or more therapies of interest), recording functions (e.g., to record device operation or status). The electronics manage and establish switchable connections between the various functional combinations or subsets of the device. For example, the electronics open and close electrical connections between the power source 206 and the control unit 214 of the NS device 102 regardless of which device sub-unit or sub-unit the power source 206 and control unit 214 are located. As other examples, the electronics open and close electrical connections between i) the telemetry component 152 and the control unit 214, ii) the energy management components 208 and 256 and the power source 206, iii) the energy management components 208 and 256 and the electrodes 112a-f (e.g., during delivery of stimulation), iv) the control unit 214 and electrodes 112a-f (e.g., during sensing operations) and the like.

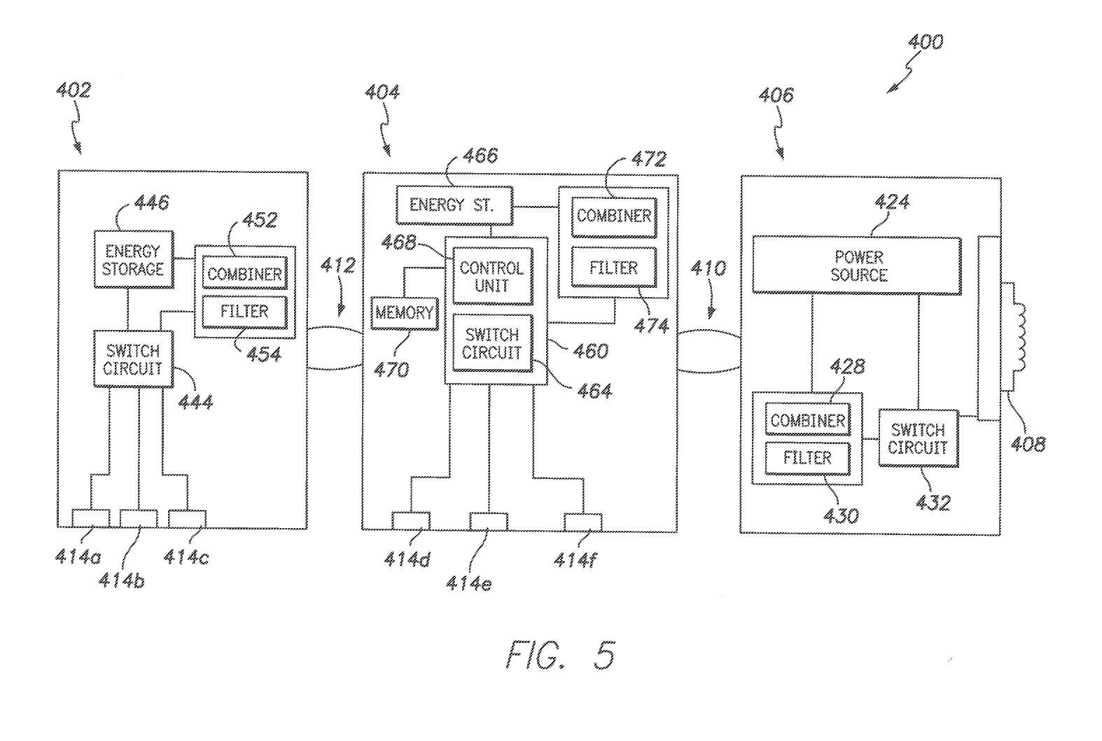

FIG. 5 is a schematic diagram of an NS device 400 in accordance with one embodiment, which may be used with an NS system. The NS device 400 includes three sub-units 402, 404, and 406 each hermetically sealed relative to one another similar to the sub-units 140 and 150. The sub-unit 404 is configured to instruct operation functions and/or settings of the sub-units 402 and 406 through the flexible inter-connects 412 and 410. The flexible inter-connects 410 and 412 physically and electrically interconnect, via a single conductive path feed-through, the sub-units 402 and 406, respectively, to the sub-unit 404. Similar to the NS device 102, each conductive path within the flexible interconnect 410 and 412 of the NS device 400 defines, respectively, a conductive path between the sub-units 402 and 404 and the sub-units 404 and 406 carrying electric charges or signals (e.g., the signal 300) that enter or leave the hermetically sealed interior of the sub-units 402, 404 and 406, respectively.

The sub-unit 406 includes a power source 424 sub-unit such as a rechargeable battery (e.g., LIR battery). The power source 424 provides power or supply voltage for the sub-unit 406 and sub-units 402 and 404 (e.g., via the device power signal 314). The sub-unit 406 is illustrated without an electric sub-system, memory, or electrodes (as illustrated in FIG. 4) allowing more space for a larger power source 424 relative to having the electric; sub-system (e.g., sub-unit 140). The larger power source 406 may increase operation time of the NS device 400 relative to a smaller power source.

The sub-unit 406 may also include a telemetry component 408 (e.g., inductive coil). The telemetry component 408 may be operatively coupled to the control unit 468 via the power/data filter 430 and power/data combiner 428, which are electrically coupled to the flexible inter-connect 410. The telemetry component 408 is configured to receive signals from an external device (e.g., the monitoring system 104) and communicate the signals to the control unit 468. The power/data combiner 428 may superimpose the received signal from the external device with the device power signal (e.g., device power signal 314) from the power source 424 carried by the flexible inter-connect 410. Once received, the control unit 468 may instruct the switching circuitry 432 to electrically couple the telemetry component 408 to the power source 424, which will receive the battery charging energy from the telemetry component 408. Optionally, the switching circuitry 432 may determine whether the external device is transmitting battery charging energy by monitoring the voltage/current of the received signal against a predetermined threshold. For example, the predetermined threshold may be a set voltage above the device power signal 314 indicating the battery charging energy. Additionally or alternatively, the telemetry component 408 may be limited to receive only battery charging energy with alternative inductive coils or telemetry components coupled to sub-units 402 and/or 404 for data communication between the external device and the NS device 400.

The sub-unit 402 has a similar component configuration as the first sub-unit 140 (e.g., energy management components 466, electric sub-system 460, control unit 468, switching circuitry 464, power/data combiner 472, a power/data filter 474, electrodes 414d-f). Further, the sub-unit 402 has a similar component configuration as the second sub-unit 140 (e.g., energy management components 446 switch circuitry 444 a power/data combiner 452, a power/data filter 454, electrodes 414a-c). The control unit 468 may control the respective switching circuitries 464 and 444 and/or stimulation pulses to generate electric pulses or emit current in accordance with parameters specified by one or more neurostimulation parameter sequences (or protocols) stored within memory 470.

For example, the neurostimulation parameter sequence, accessed by the control circuit 468, requires the electrode 414b to transition from an anode to a cathode configuration to stimulate a pulse. The control circuit 468 transmits the command (e.g., sequence of bits) to the power/data combiner 472 with instructions to transmit along the flexible inter-connect 412. Optionally, the command may include an address representing the intended recipient, e.g., sub-unit 402. The power/data combiner 472 may superimpose or embed the command (e.g., embedded signal 312) with the device power signal (e.g., 314) configuring a signal (e.g., 300) carried along the flexible inter-connect 412. The signal may be received by a power/data filter 454, which partitions the device power signal from the command or embedded signal. Switch circuitry 44 receives the command from the power/data filter 454 and transitions the stage of the electrode from an anode to a cathode by sinking the electrode to ground.

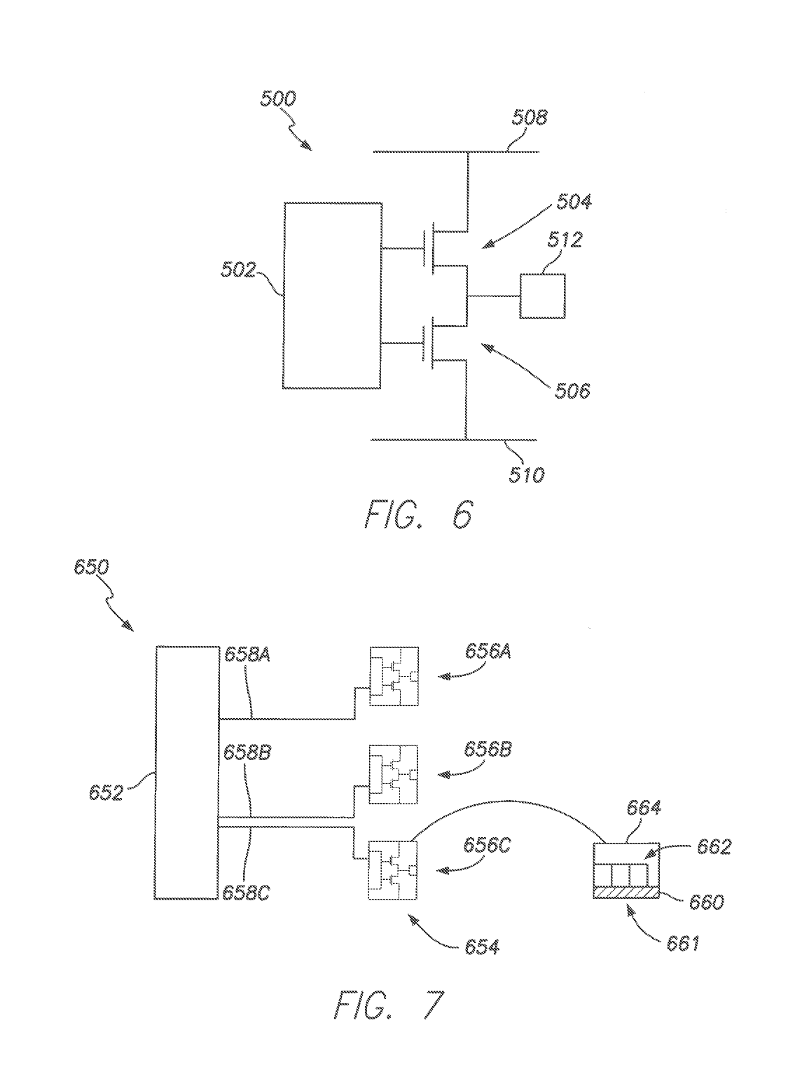

FIG. 6 illustrates a single representative cell 500 for controlling an operating state of an electrode, such as the electrodes 414a-f (FIG. 4). The cell 500 may be implemented within a multiplexer or other switching circuitry, such as the switching circuitry 212 (FIG. 2) or 444 (FIG. 4), and may be electrically coupled to multiple electrodes. The cell 500 may also be implemented within a single electrode such that, in some embodiments, at least some of the electrodes of the plurality of electrodes (e.g., 112) may contain the cell 500. The cell 500 includes logic circuitry 502 that is configured to control transistors 504, 506 in accordance with a designated sequence or protocol. The transistor 504 is electrically coupled to a power line 508 and is configured to receive electrical current therefrom. The transistor 506 is electrically coupled to a ground line 510. An output 512 is electrically coupled between the transistors 504 and 506. The output 512, in turn, may be electrically coupled to the electrode (not shown).

As set forth herein, the electrodes may be configured to have at least two operating states. In particular embodiments, the electrodes are configured to have one of three operating states. The operating states may be a source state such that the electrode functions as an anode, a sink state such that the electrode functions as a cathode, or an inoperative or inactive state such that the electrode effectively does not supply or draw current. For example, when the electrode is in the source state, the transistor 504 may be dosed and the transistor 506 may be open such that current flows from the power line 508 through the circuitry to the output 512. When the electrode is in the sink state, the transistor 504 may be open and the transistor 506 may be dosed such that current flows from the output 512 to the ground line 510. When the electrode is in an inoperative state, each of the transistors 504, 506 are opened. In such embodiments, the cell 500 has a high impedance such that current does not effectively flow through the output 512 in either direction. It is noted, however, that the cell 500 is only representative of how circuitry may be configured to control the operating state of an electrode. Other circuits may be used in other embodiments.

In some embodiments, the switching circuitry 212, 258, 444, or 464 may have a plurality of the cells 500 therein. In such embodiments, each of the outputs 512 may be electrically coupled to one of wire conductors coupled to the electrode. Accordingly, the output 512 may be selectively controlled to supply or draw current through the respective wire conductor or to effectively render the wire conductor inoperative with high impedance. For example, the sub-unit 140 in FIG. 2 includes three electrodes 112a, 112b, and 112c that receive current directly from the switching circuitry 212. As such, the switching circuitry has three wire conductors.

FIG. 7 illustrates an electrical diagram 650 for generating electric pulses proximate to nervous tissue in accordance with an embodiment. As shown, switching circuitry 652 is electrically coupled to electrodes 654. The electrodes are arranged in columns 656A-656C. In FIG. 6, only a single electrode 654 is shown in each column, but it is understood that each column may include more than a single electrode. Each of the electrodes 654 of a single column is electrically coupled to the switching circuitry 652 through a common (i.e., the same) control line. For example, the electrodes 654 of the column 656A are electrically coupled to a control line 658A, the electrodes 654 of the column 656B are electrically coupled to a control line 655B, and the electrodes 654 of the column 656C are electrically coupled to a control line 658C. Although not shown, each of the electrodes may be electrically coupled to a power line and a ground line. The power and ground ones may electrically couple to a combination of the electrodes 654.

In some embodiments, more than one control line may be electrically coupled to the electrodes of a column, and the electrodes may be controlled in accordance with a designated protocol. For example, the electrode 654 of the column 656A may be electrically coupled to two control lines. A first control line may be a data line and a second control line may be a clock line. By way of example only, the first and second control lines may be operated in accordance with inter-integrated circuit protocol (or I2C protocol). The switching circuitry 652 is configured to communicate control signals through the control line 658A. The control signals may represent, among other things, operating states of the electrodes 654.

A side view of a representative electrode 654 is also shown in FIG. 6. The electrodes 654 may include a housing 664 (e.g., a ceramic housing) that is configured to hermetically seal internal circuitry of the electrode 654. The housing 664 may be mounted to a stimulating element 660 that is electrically coupled to logic circuitry 662. The logic circuitry 662 may be disposed within a cavity formed by the housing 664. The logic circuitry 662 may be electrically coupled to one of the control lines, a ground line (not shown), and a power line (not shown). The logic circuitry is also coupled to the stimulating element 660 for controlling the operating state of the stimulating element 660. The stimulating element 660 includes a circular surface 661 that is configured to interface with the nervous tissue.

The logic circuitry 662 is configured to receive control signals (e.g., from the switching circuitry 652) through the corresponding control line and identify the instructed operating state for the corresponding electrode 654 based on the address that is designated to the electrode 654. In response to the control signals, the logic circuitry 662 in the electrode 654 of column 656A may change or maintain the operating state. During operation, each of the electrodes 654 is capable of drawing power from a power line to operate as a source or using a ground line to operate as a sink. In such embodiments, fewer wire conductors may be used than embodiments that utilize a single wire conductor for each electrode.

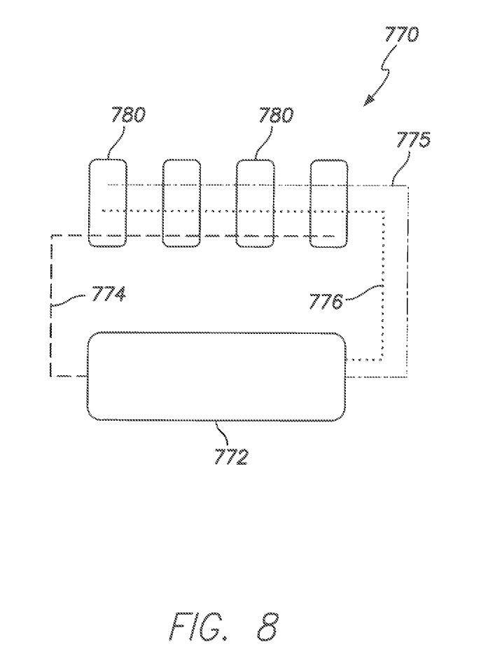

FIG. 8 illustrates another electrical diagram 770 for generating electric pulses proximate to nervous tissue in accordance with an embodiment. As shown, the electrical diagram 770 includes switching circuitry 772, a plurality of wire conductors 774-776, and electrodes 780. The wire conductors include a power line 774, a ground line 775, and a control line 776. Each of the power line 774, the ground line 775, and the control line 776 is electrically coupled to each of the electrodes 780 in FIG. 7. Although four electrodes 780 are shown in FIG. 7, embodiments may include fewer or more electrodes (e.g., one, two, three). During operation, each of the electrodes 780 is capable of drawing power from the power line 774 to operate as a source or using the ground line 775 to operate as a sink. Similar to above, the switching circuitry 772 may broadcast control signals that represent addresses and operating states associated with the addresses. Each of the electrodes 780 may be designated with one of the addresses and may be configured to identify the operating state associated with the respective address. Accordingly, in some embodiments, the switching circuitry 772 may be capable of selectively operating the electrodes using only three wire conductors.

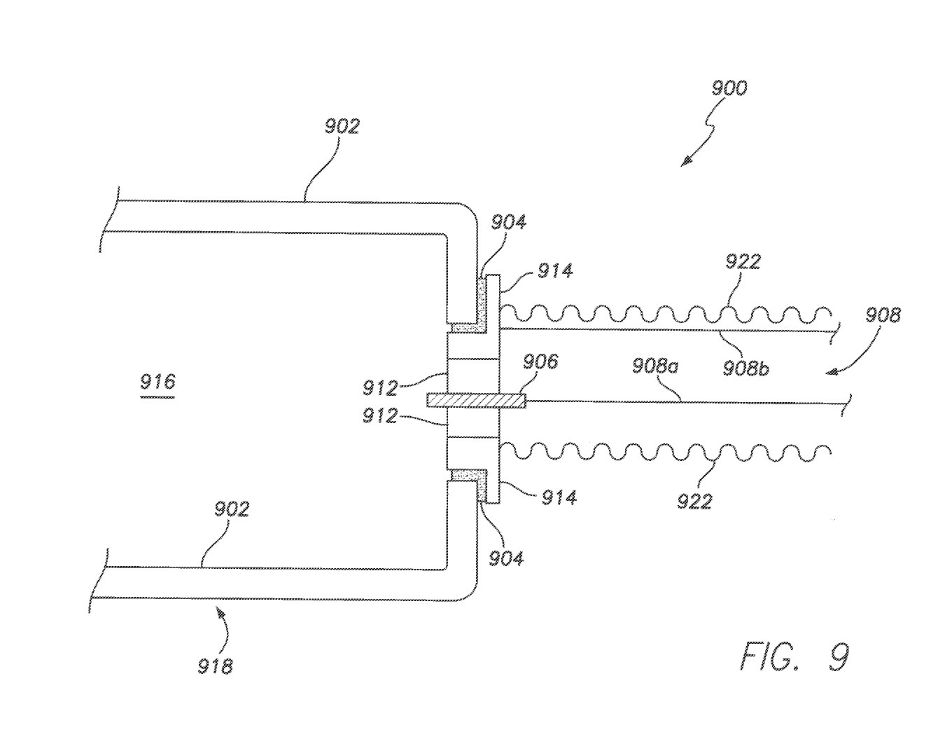

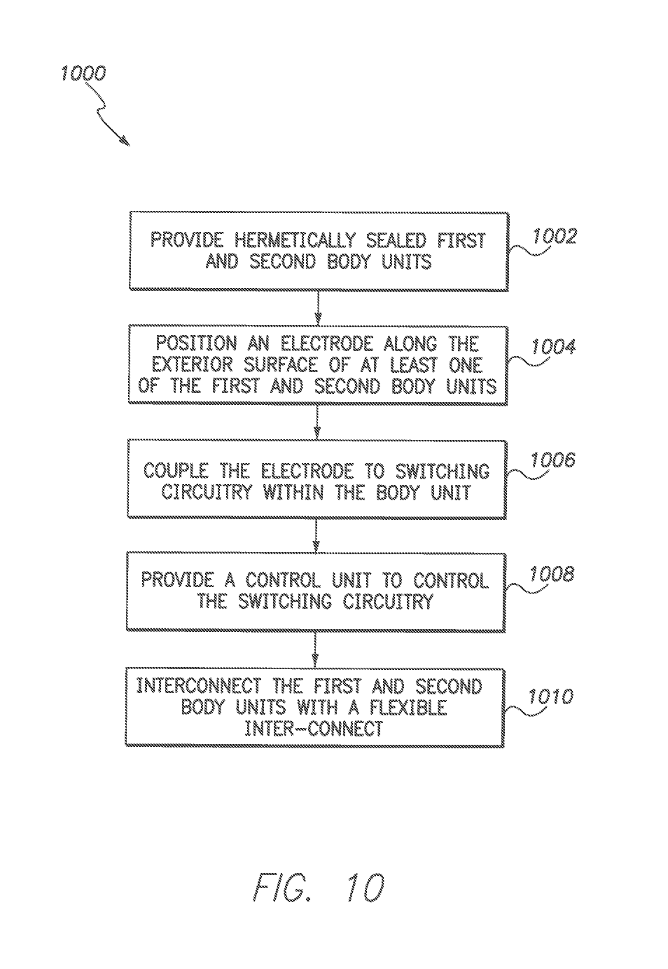

FIG. 9 illustrates a schematic cross sectional drawing 900 of a flexible inter-connect 908 of a sub-unit 918 in accordance to an embodiment. The sub-unit 918 may be similar to sub units 140, 150, 402, and/or 406. It should be noted that the internal components (e.g., control unit, switching circuitry, energy management components) are not shown. The inter-connect 908 may include two flexible conductive paths a common ground 908b (similar to the common ground 108b) and a conductive path feed-through 908a (similar to the conductive path feed-through 108a).

The conductive path feed through 908a is electrically coupled to a feed-through 906. The feed-through 906 defines a conductive path into and out of an interior 916 of the sub-unit 918. The feed-through 906 may be surrounded by an insulator 912 (e.g., ceramic, glass, plastic) positioned between the feed-through 906 and a cylindrical flange 914. The flange 914 may be coupled to a can 902 of the sub-unit 918 at a hermetic seal 904. The hermetic seal 904 may be a filler metal (e.g., solder) or a welding joint coupling the flange 914 to the can 902. The hermetic seal 904 and the insulator 912 hermetically seal the sub-unit 918 surrounding the feed-through 906 providing a conductive path entering or leaving the sealed interior 916 of the sub-unit 918. The flange 914 and the can 902 may be made from a corrosive resistant material (e.g., titanium, gold), which further allows electrical charge to flow between the can 902 and the flange 914. The common ground 908b may be coupled to the flange 914. The common ground 908b is electrically coupled to the can 902 via the hermetic seal 904. Optionally, the common ground 908b and the conductive path feed-through 908a may be surrounded or embedded within a flexible insulator 922, such as silicon rubber, electrically isolating the common ground 908b and the conductive path feed-through 908a from each other or external tissue. Optionally, the flexible insulator may be partitioned into two pieces each with one of the common ground 908b or the conductive path feed-through 908a. Additionally or alternatively, the conductive path feed-through 908a may be surrounded or embedded within the flexible insulator 922 and the common ground 908b may not be surrounded by the flexible insulator 922.

FIG. 10 is a flowchart illustrating a method 1000 of manufacturing an NS device. The method 1000, for example, may employ structures or aspects of various embodiments (e.g., systems and/or methods) discussed herein. For example, the NS device may be similar to the NS device 102 (FIGS. 1 and 2) or the NS device 400 (FIG. 4) or may include other features, such as those described or referenced herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. Furthermore, it is noted that the following is just one possible method of manufacturing an NS device. Other methods may be used.

The method 1000 includes providing (at 1002) hermetically sealed first and second sub-units. For example, the first and second sub-units 140 and 150 described above, which include one or more power sources 206 and one or more energy management components 208 and 256 electrically coupled to the power source 206.

The method 1000 includes positioning (at 1004) at least one electrode, along the exterior surface of at least one of the first and second sub-units. For example, electrodes 112a-f may be in the shape of a ring that continuously covers the circumference of the exterior surface of the first and second sub-units 140 and 150. The ring shape of the electrodes 112a-f allows the electrodes 112a-f to emit current or generate electric pulses in an outward radial direction proximate to the nervous tissue.

The method 1000 includes coupling (at 1006) the electrode 112a-f to the switching circuitry 212 and 258 within the sub-unit. Additionally, the method 1000 includes providing (at 1008) the control unit 214 to control the switching circuitry. For example, the switching circuitries 212 and 258 may be coupled to the electrodes through the common control lines 658A-C. The common control lines 658A-C is used by the switching circuitries 212 and 258 to set the electrode 112a-f to one of the states (e.g., anode, cathode, open). The control unit 214 may be within the same sub-unit as the switching circuitry (e.g., switching circuitry 212) and/or the control unit may be within an alternative sub-unit (e.g., switching circuitry 258). The control unit 214 may control the respective switching circuitries 212 and 258 to generate electric fields or emit current in accordance with parameters specified by one or more neurostimulation parameter sequences (or protocols) stored within the memory 204.

The method 1000 includes interconnecting (at 1010) the first and second sub-units with a flexible inter-connect 108. For example, the flexible inter-connects 410 and 412 physically and electrically interconnecting (via a single conductive path feed-through) the sub-units 402 and 406, respectively, to the sub-unit 404. In another example, the flexible inter-connect 108 electrically interconnects the internal components of the first and second sub-units 140 and 150 (e.g., the power source 206, the electronics sub-system 202, energy management components 208 and 256, and telemetry component 152) via the single conductive path feed-through 108a.

The control units 214 and 468 may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the control units 214 and 468 may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The control units 214 and 468 may execute a set of instructions that are stored in one or more storage elements (e.g., memory 204 and 470), in order to process data. The storage elements may also store data or other information as desired or needed. The storage element may be in the form of an information source or a physical memory element within the control units 214 and 468. The set of instructions may include various commands that instruct the control units 214 and 468 to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f), unless and until such claim limitations expressly use the phrase “means for” followed by a statement of function void of further structure.

What is claimed is:

1. A method for manufacturing a leadless neurostimulation (NS) device to be implantable proximate to a spinal column of a patient, the method comprising:
providing hermetically sealed first and second sub-units; wherein the first and second sub-units comprise a power source and an energy management components coupled to the power source;
positioning at least one electrode along the exterior surface of at least one of the first and second sub-units, wherein the electrode is configured to generate an electric pulse in an outward radial direction proximate to nervous tissue;
coupling the electrode to a switching circuitry configured to electrically set a state of the electrode;
providing a control unit in at least one of the first and second sub-units, the control unit configured to execute a protocol determining the state of the electrode;
interconnecting the first and second sub-units with a flexible inter-connect, wherein the flexible inter-connect includes a single conductive path feed-though located at a first end of the first sub-unit and a first end of the second sub-unit, the single conductive path feed-through is configured to carry at least two of device power, communication data, and stimulation pulses between the first and second sub-units.
